(19) United States
(12) Patent Application Publication
(10) Pub. No.: US 2010/0227414 A1
(43) Pub. Date: Sep. 9, 2010

(54) Affinity capture mass spectroscopy with a porous silicon biosensor

(75) Inventor: John Lawrence Ervin, San Diego, CA (US)

Correspondence Address: TREX ENTERPRISES CORP., 10455 PACIFIC COURT, SAN DIEGO, CA, 92121, US

(73) Assignee: Trex Enterprises Corp.

(21) Appl. No.: 12/660,877

(22) Filed: Mar. 5, 2010

Related U.S. Application Data
(60) Provisional application No. 61/209,416, filed on Mar. 5, 2009.

(51) Int. Cl. G01N 33/566

(52) U.S. Cl. 436/501

(57) ABSTRACT

Affinity Capture-Mass Spectroscopy (AC-MS), an analytical technique which couples the sensitivity of a label-free binding detected biosensor, and the information richness of mass spectroscopy is described. A 3-dimensional porous silicon bio-surface is used to capture proteins, DNA, or small molecules while acquiring a label-free, time resolved signal linearly proportional to the amount of binding. A switch to dissociative buffer conditions then frees the captured molecule for analysis by mass spectroscopy. In particular, techniques for use with electrospray mass spectroscopy are described.

### CROSS-REFERENCE TO RELATED APPLICATIONS

This application claims the benefit of Provisional Application Ser. No. 61/209,416 filed 03-05-2009, entitled Mass Spectroscope with Porous Silicon Flow Cell.

### FIELD OF INVENTION

This invention relates to mass spectroscopy and optical biosensors and in particular to porous silicon biosensors.

### BACKGROUND OF THE INVENTION

### Optical Biosensors

An optical biosensor is an optical sensor that incorporates a biological sensing element. In recent years optical biosensors have become widely used for sensitive molecular binding measurements. To study interactions of proteins with other biomolecules one may generally use labeled or label-free methods. For these methods a first molecule of interest (a receptor, also referred to as a ligand) is immobilized onto a surface. An interaction is monitored by then introducing additional molecules (a target, also referred to as an analyte) and detecting whether they in fact bind to the receptor. When using labels to monitor these interactions a fluorescent, colorimetric or some other signal is generated by an additional molecule or moiety that is attached to the target or receptor which gives a signal when the interaction takes place. This so called label (or tag) is present only to detect the interaction and is not part of the interaction of interest per se.

In label-free binding, on the other hand, the receptor and target binding are monitored directly using untagged biomolecules. A variety of technologies exist in the art to detect binding without labels including surface plasmon resonance (SPR) and white light interferometry using porous silicon. In addition to the variety of technologies which exist to monitor label-free binding events, there are a variety of instrument architectures which can used. These include plate readers and flow cells. In the case of plate readers a well plate (or micro well plate or micro titer plate) is used to house the biochips and fluids which are used for the label-free binding studies. This allows for parallel analyses of several types of data. Alternatively flow cells house biochips in, typically, a microfluidic cell which routes fluid over the region of the biochip where the binding interaction takes place.

When acquiring and analyzing data of this sort there are a number of steps which are performed for the data analysis (the data method) on a number of channels (be those channels, flow cells or wells in a well plate). A file format which captures the full gamut of what a user of the analytical instrument might want to do must incorporate flexibility in acquisition and in analysis.

### Kinetic Binding Measurements

Kinetic binding measurements involve the measurement of rates of association (molecular binding) and disassociation. Analyte molecules are introduced to ligand molecules producing binding and disassociation interactions between the analyte molecules and the ligand molecules. Association occurs at a characteristic rate [A][B]kₒₙ that depends on the strength of the binding interaction kₒₙ and the ligand topologies, as well as the concentrations [A] and [B] of the analyte molecules A and ligand molecules B, respectively. Binding events are usually followed by a disassociation event, occurring at a characteristic rate [A][B]k_{off} that also depends on the strength of the binding interaction. Measurements of rate constants kₒₙ and k_{off} for specific molecular interactions are important for understanding detailed structures and functions of protein molecules. In addition to the optical biosensors discussed above, scientists perform kinetic binding measurements using other separations methods on solid surfaces combined with expensive detection methods (such as capillary liquid chromatography/mass spectrometry) or solution-phase assays. These methods suffer from disadvantages of cost, the need for expertise, imprecision and other factors.

### Surface Plasmon Resonance

An optical biosensor technique that has gained increasing importance over the last decade is the surface plasmon resonance (SPR) technique. This technique involves the measurement of light reflected into a narrow range of angles from a front side of a very thin metal film producing changes in an evanescent wave that penetrates the metal film. Ligands and analytes are located in the region of the evanescent wave on the backside of the metal film. Binding and disassociation actions between the ligands and analytes can be measured by monitoring the reflected light in real time. These SPR sensors are typically very expensive. As a result, the technique is impractical for many applications.

There have been attempts to combine SPR with mass spectroscopy (SPR-MS, see below) but these invariably suffer from the low capture capability of the planar SPR surfaces and could not be directly coupled to an electrospray mass spectrometer as described here with AC-MS. Also, SPR typically involves monitoring light that has undergone total internal reflection in a prism. This prism requirement has generally precluded SPR from being economically viable on a per-well basis. That is, SPR has not been economically adapted to measuring data directly in a microtiter well plate.

### Porous Silicon Layers

U.S. Pat. No. 6,248,539 (incorporated herein by reference) discloses techniques for making porous silicon and an optical resonance technique that utilizes a very thin porous silicon layer within which binding reactions between ligands and analytes take place. The association and disassociation of molecular interactions affects the index of refraction within the thin porous silicon layer. Light reflected from the thin film produces interference patterns that can be monitored with a CCD detector array. The extent of binding can be determined from change in the spectral pattern. Prior art proposed techniques for utilizing porous silicon for optical analysis include a micro-well format where porous silicon chips positioned in wells of micro-well plates or they propose a format in which the chips are positioned in a flow cell through which the fluids containing the ligands and analytes flow.

### Nano-Pore Optical Interferometry

Martin et al. (U.S. Pat. No. 7,517,656, incorporated herein by reference) teach the use of nano porous silicon coupled with white light interferometry, giving a label-free biosensor capable of sensitively measuring binding interactions between biomolecules. This technique, called NPOI (nano-pore optical interferometry) will be extended here. The details of the porous silicon substrate appropriate for this invention are taught by Rauh-Adelmann et al. (application Ser. No. 11/180,394, publication number US 2007/0012574 A1, incorporated herein by reference) and the details of the surface preparation of the porous silicon to be used is taught by Ervin et al. (application Ser. No. 12/221,129, publication US2009/0036327A1, incorporated herein by reference). With regard to porous silicon sensors, Ghadiri, et al. (U.S. Pat. No. 6,720,177) teach the use of a reflective porous silicon substrate for biosensing using optical detection and further in a division of the aforementioned application (U.S. Pat. No. 6,897,965 B2) teach the use of porous silicon formed in the form of a Fabry-Perot cavity for use as a biosensor, again with interferometric readout. All of these patents are incorporated herein by reference.

NPOI measurements, as opposed to other label-free binding detected biosensor techniques, may be taken using instrumentation designed for measuring samples in flow cells or directly in micro-titer well plates. The design of the instrumentation for these dual format measurements is taught by Ervin et al. (application Ser. No. 12/221,119) and specifically for the measurement in micro-titer plates is taught by Ervin et al as well (application Ser. No. 12/221,182).

### Flow Cell Measurements

In flow cell measurements using NPOI the porous silicon chip is placed into a flow cell cartridge where fluidic channels gasketed against the porous silicon chip itself form a fluidic unit called a flow cell. Here pumps and valves external to this formed flow cell, direct a series of aqueous, organic; or aqueous/organic fluids containing, for instance but not limited to, buffers, reagents, proteins, small molecules or DNA, across this chip, typically in an automated sequence. Flow cells are particularly suited for time resolved measurements as rapidly flowed fluids ensure a fresh supply of reagent at a known concentration, and off board valves, ensure rapid transition between conditions. In particular when starting an association reaction valves actuate the transition in less than 1 second, likewise with dissociation.

### Plate Reader Measurements

Microtiter well plates are ubiquitous in biochemical research. Standards exist for their footprint and for the individual well pitch, and standard configurations exist for both well numbers and well volumes. In the context of NPOI described here, plate reader measurements refer to measurements in standard microtiter well plates using porous silicon, biosensor chips. Measurements of this sort are particularly suited for medium to high-throughput contexts where many samples need to be measured to understand, for instance their concentration, binding kinetics or binding affinities, but the precision of flow cell measurements is not required. These types of measurements are often performed in, for instance an antibody process context where production biochemists are interested in the concentration of a particular antibody molecule secreted into a complex medium containing many other molecules. Porous silicon biochips are treated to only capture the antibody molecule of interest, and NPOI is used to measure the concentration of the uniquely captured antibody.

### Separations-Based Measurements

More recently, optical biosensors have been used as an alternative to conventional separations-based instrumentation and other methods. Most separations-based techniques have typically included 1) liquid chromatography, flow-through techniques involving immobilization of capture molecules on packed beads that allow for the separation of target molecules from a solution and subsequent elution under different chemical or other conditions to enable detection; 2) electrophoresis, a separations technique in which molecules are detected based on their charge-to-mass ratio; and 3) immunoassays, separations based on the immune response of antigens to antibodies. These separations methods involve a variety of detection techniques, including ultraviolet absorbance, fluorescence and even mass spectrometry. The format also lends itself to measure of concentration and for non-quantitative on/off detection assays.

### Thin Films

It is well known that monochromic light from a point source reflected from both surfaces of a film only a few wavelengths thick produces interference fringes and that white light reflected from a point source produces spectral patterns that depend on the direction of the incident light and the index of refraction of film material. (See “Optics” by Eugene Hecht and Alfred Zajac, pg. 295-309, Addison-Wesley, 1979.)

### Mass Spectroscopy

Mass spectroscopy is a well-known analytical technique which is used to measure the mass to charge ratios of molecules in the gas phase. It can be used to identify both small molecules, taken here to be molecules, whose molecular weight is between 100-1000 Da, and to identify peptides and proteins. This technique takes many different forms that, in the context here, are often differentiated by the technique used to ionize molecules into the gas phase, by the technique used to detect molecules, and whether tandem mass spectroscopy is in use. In the work described here liquid samples were introduced into the mass spectroscopy using electrospray ionization, including nano-spray. Trap, quadropole and time of flight ion detection schemes where all tested.

Often mass spectroscopy is performed with a liquid chromatography step on the front end. This well known analytical technique is called liquid chromatography-mass spectroscopy (LC-MS). Here the initial LC separation is affected to separate the molecules to be analyzed so that they are not all introduced into the mass spectrometer at the same time. With the invention here, this is useful for separating buffer salts from small molecules which after capture and release, separating small molecules from each other after capture and release, separating peptides from one another and buffer salts after capture, release and digest, and separating intact proteins and peptides from one another after capture and release.

### Affinity Capture

Affinity chromatography is a well known technique which separates molecules based on their relative affinity, or binding strength, for another molecule. In a chromatographic context, a stationary phase is formed by either covalently or non-covalently linking a ligand molecule to a stationary phase, often polymeric beads. The mobile phase typically contains a mixture of analytes with different binding affinities for that stationary ligand and is flowed across the stationary phase. Molecules which bind to the stationary ligand elute more slowly than those that do not.

In affinity capture, ligands are likewise immobilized, but have affinities strong enough that they may be bound long enough, that most to all other non-binding molecules may be separated from the captures molecule or molecules. These molecules are then eluted into a different direction than the other non-captured molecules. It differs from chromatography in that in chromatography all molecules, whether captured or not flow the same direction, whereas in affinity capture, bound molecules are held, and then eluted in a different direction than those which are not bound.

### Knowledge of the Analyte

Biosensors are capable of exquisite characterization of the binding and unbinding rates of pairs of molecules. However, this information is generally relevant because a researcher knows what the molecules of interest are before they are placed into the biosensor. Biosensors in this sense are sensitive, in that they are able to detect binding between analytes in solutions and ligands on surfaces with limits of detection often in the pg/mL level. But an experiment where, for instance, the analyte flowing across an immobilized ligand is not known, one could still measure the kinetic constants and affinity of the measurement, but typically the biosensor instrument could not identify the compound. The biosensor needs to be coupled to an information rich detector, like a mass spectrometer to make that determination. However, for a proper, fully integrated system, the affinity capture biosensor, must capture enough material to realize a quality signal on the mass spectrometer. This has historically limited the direct coupling of label-free biosensor to electrospray mass spectroscopy and is addressed here by the use of porous silicon which has a large enhancement of surface area to sensor area.

While, Dollinger, et al. (U.S. Pat. No. 5,891,741 ‘Affinity Selection of Ligands by Mass Spectroscopy’) teach the use of mass spectroscopy for studying relative affinities of two molecules for a target moiety, they provide no label-free binding signal to characterize the kinetics of the interaction in their invention. However Siuzdak, et al. (U.S. Pat. No. 6,288,390 B1) teach the use of porous silicon as a matrix replacement for matrix-assisted laser desorption/ionization (MALDI) spectroscopy. Here porous silicon is used in concert with mass spectroscopy, as in this invention, however Siuzdak, et al. are nor providing biosensor data, nor are they providing an interface to electrospray ionized mass spectroscopy in their invention. Finally, Nelson et al. (U.S. Pat. No. 5,955,729) teach the use of Surface Plasmon Resonance-Mass Spectroscopy (SPR-MS). Nelson et al. do teach the coupling of a label-free biosensor to mass spectroscopy, but only do so using the SPR technique for biosensing. This technique suffers from the low binding capacity of the planar SPR surface and has generally been limited to only MALDI detected mass spectroscopy in practice due to this limited capability. Nelson et al. do not teach the coupling of NPOI with mass spectroscopy in both plate reading and flow cell modes.

### The Need

What is needed is a label-free process for making molecular ligand-analyte binding measurements when the analyte is unknown.

### SUMMARY OF THE INVENTION

The present invention is an analytical process for making molecular ligand-analyte binding measurements when the analyte is unknown. The process couples the sensitivity of a label-free binding detected biosensor, and the information richness of mass spectroscopy. A 3-dimensional porous silicon bio-surface is used to capture proteins, DNA, or small molecules while acquiring a label-free, time resolved signal linearly proportional to the amount of binding. A switch to dissociative buffer conditions then frees the captured molecule for analysis by mass spectroscopy. In particular, techniques for use with electrospray mass spectroscopy are described.

Porous silicon is suitably prepared to be a substrate capable of both revealing the nano-pore optical interferometric (NPOI) signals as well as affinity capturing enough material to be suitably analyzed by mass spectroscopy down stream of the NPOI instrumentation. The technique can be utilized in a flow cell mode or in a plate reader mode based NPOI characterization.

For flow cell based methods, a bait molecule (the ligand) is tethered to the porous silicon based sensor chips using covalent methods, non-covalent methods, or a combination of covalent and non-covalent methods. Samples, containing molecules (the analyte) which may bind to the bait molecule, are then flowed across the immobilized bait. Molecules which bind with an appreciable affinity are then themselves temporarily immobilized on the chip, whereas those which do not flow into waste. After trapping these ‘prey’ molecules, conditions are then set to elute these molecules into a mass spectrometer. Often, in particular with small molecules, this dissociation of the analyte from the bait is spontaneous, other times, like in DNA binding or for instance antibody, antigen interactions conditions are altered chemically to affect the dissociation by, for instance, lowering pH or adding surfactant.

With flow cell instrumentation, three schemes are described for coupling to electrospray ionization, including nano-spray and atmospheric pressure, chemical ionization (APCI), and one to matrix assisted laser desorption (MALDI) mass spectroscopy. In the first electrospray scheme, the sample is fraction-collected during the dissociation step and then subsequently introduced into the mass spectrometer. This scheme would be used, in for instance ‘bottom up’ proteomics research. Here, protein is collected and then digested off line, in for instance a trypsin containing digest media. The tryptic peptides are then separated and analyzed by liquid chromatography-mass spectroscopy (LC-MS).

In the second electrospray scheme, the sample is captured, but now directly eluted into the mass spectrometer for analysis. This could be used, for instance in the case of peptides which are screened in large numbers in the search for a binder to an immobilized protein. The third electrospray scheme uses a trap and elute scheme to capture biomolecules on the porous silicon, affinity capture surface. These molecules are then released and then trapped on a chromatographic trap column. These molecules are then released into the mass spec using LC-MS and analyzed. This scheme could be used, for instance in a small molecule discovery context. Here, typically several 10s to several 100s of molecules are introduced over a porous silicon affinity capture surface prepared with the protein of interest. Bound molecules are then separated from unbound molecules and these bound molecules are trapped in the trap column, and eluted and analyzed by LC-MS. In this way, small molecule binders may be efficiently discovered from a library. In the fourth electrospray scheme, time resolved information about the dissociation is gathered, by either trapping in a chromatographic trap column as in the third scheme or fraction collecting as in the first scheme. The idea with time resolved AC-MS is to generally characterize the rate of dissociation. In this way a two or more point dissociation curve may be gathered. This could be used, in for instance, a molecular fragment characterization. Here a small molecule fragment, say weighing only 120 Da, could undergo careful off rate characterization. Molecules or molecule fragments below 250 Da are difficult to analyze by label-free binding techniques that use optical detection means, as these molecules inherently cause a small change in signal and are often dissolved in organic/buffer mixtures which obscure the signal. In particular low molecular weight carbohydrate molecules are nearly impossible to analyze as these molecules have very similar refractive indices to water and therefore give next to no signal change upon binding in typical label-free instrumentation, like surface plasmon resonance (SPR). By using the mass spectrometer as the detector, in this time resolved AC-MS scheme, the low molecular weight range limitation disappears during off rate characterization.

Using flow cell based instrumentation, coupled with MALDI mass spectroscopy, time resolved dissociation may be characterized. Here a MALDI spotter is used in a technique similar to the time-resolved fraction collection scheme described above. The spotter uses time sliced segments from a spontaneous dissociation segment of the binding reaction. Spots are progressively formed on a spotting plate where the order in which they are formed is associated with the time during the dissociation. The spotter may add matrix to the dissociating eluent from the NPOI instrument in order to later affect MALDI, for instance 2,5-dihydroxy benzoic acid (DHB), or the spotter may spot onto a matrix that itself absorbs light to affect the MALDI, for instance onto the desorption ionization on silicon (DIOS) plates sold by Waters Corporation, Milford, Mass.

A user skilled in the art will recognize the many uses for the AC-MS embodiments described here.

### BRIEF DESCRIPTION OF THE DRAWINGS

FIG. 1 shows the prior art NPOI scheme for analyzing biomolecular interactions without labels in either a flow cell or plate reader scheme.

FIG. 2 shows the two-channel, prior art fluidic diagram of the multiple format biosensor instrument generally used in flow cell embodiments of NPOI described here.

FIG. 3 shows an AC-MS embodiment using a flow cell NPOI instrument, an electrospray, nanospray or APCI sourced MS together with fraction collecting to make the AC-MS samples.

FIG. 4 shows an AC-MS embodiment using a flow cell NPOI instrument, an electrospray, nanospray or APCI sourced MS together with direct elution into the mass spectrometer.

FIG. 5 shows an AC-MS embodiment using a flow cell NPOI instrument, an electrospray, nanospray or APCI sourced MS, together with a trap and elute scheme for introducing molecules into the mass spectrometer.

FIG. 6 shows an AC-MS embodiment using a flow cell NPOI instrument, an electrospray, nanospray or APCI sourced MS, together with a trap and elute scheme for introducing molecules into the mass spectrometer.

FIG. 7 shows an AC-MS embodiment using a flow cell together with a MALDI spotter to give time resolved off rate data.

FIG. 8 shows AC-MS data taken on a protein-protein pair with an NPOI flow cell, a micro-flow electrospray ionization source, and a quadrapole, ion-trap tandem mass spectrometer using the fraction collecting embodiment described here.

FIG. 9 shows AC-MS data taken on a small molecule-protein pair with an NPOI flow cell, an electrospray ionization source, and an ion-trap mass spectrometer using the direct elution embodiment described here.

FIG. 10 shows a ‘well strip’ holding porous silicon chips suitable for use with AC-MS in well plates using an NPOI plate reader instrument.

FIG. 11 shows a typical 96-well microtiter plate preparation which would be used for AC-MS data in plates.

### DETAILED DESCRIPTION OF PREFERRED EMBODIMENTS

The invention is directed at affinity capture-mass spectroscopy (AC-MS), an analytical technique which couples the sensitivity of a label-free binding detected biosensor, and the information richness of mass spectroscopy. Described here will be various methods and apparatuses that allow for the full AC-MS and NPOI characterization of binding partners in a single run. A porous silicon biochip makes this possible.

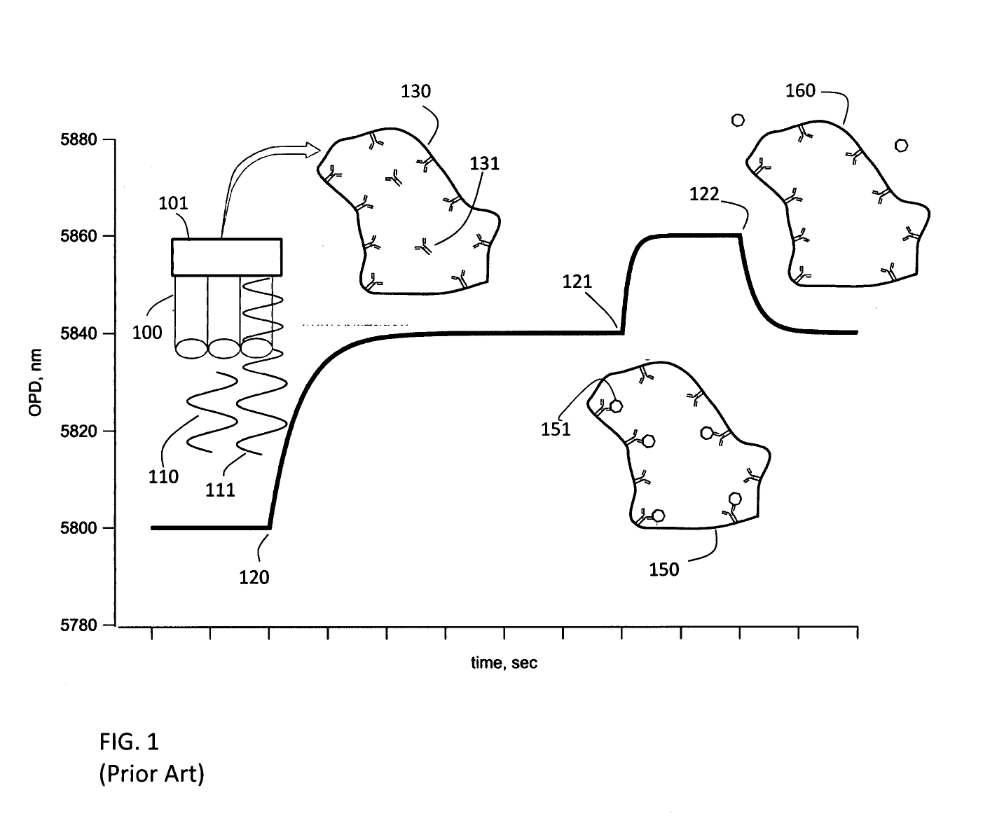

As shown in FIG. 1, a porous silicon biochip is formed by having pores 100, with average diameters of typically 80 nm, but with average diameters which may vary between 15-500 nm, etched to a depth of between 0.5-10 μm, but typically between 1.5-2.0 μm into silicon substrates 101 with thicknesses which may vary between 100-1,000 μm but are typically 200 μm. The pores formed into these substrates are of a size that allow biomolecules to freely diffuse inside of them, but are large compared to the wavelength of light used to probe these pores.

In a typical nano-pore optical interferometry (NPOI) experiment the fundamental signal is the optical path difference (OPD), which is the difference in optical length between the top surface and bottom surface reflection of the porous silicon layer used. Light partially reflects off the top surface of the porous silicon 111) because porous silicon typically has a different refractive index (n=1.8) than does, say water (n=1.3). Light also partially reflects off of the bottom surface of the porous silicon layer 111 as the porous silicon typically has a very different refractive index than does the silicon substrate (n=3.7). The interference pattern from this reflected light is analyzed as described by Martin et al. (U.S. Pat. No. 7,517,656) to give the OPD.

To use these pores and the OPD signal in a nano-pore optical interferometry experiment (NPOI), a capture molecule 131 is first tethered onto the surface of the inside of the pores 130 either covalently, non-covalently or with a mixture of covalent and non-covalent interactions in an immobilization stage 120. The prepared surface is then often washed, and binding molecules 151) are introduced in an associative stage, or binding stage 121. As molecules then bind they enter the pore 150, which displaces water, which then causes a change in OPD. As OPD is linearly proportional to the amount of material bound, the rate of change of OPD in this stage, together with information about the concentration of the binding molecule, is generally used to give information about the bimolecular on rate, kₒₙ. Typically the binding experiment concludes with a switch to dissociative buffer 122 and the bound molecules then dissociate from the initially tethered molecule 160. The rate of change in the OPD signal in this stage is then used to give the off rate, k_{off}.

Traces like those shown in the NPOI graphs of (800) and (900) may be analyzed mathematically, to derive these rates, and often give a model (e.g. bimolecular, heterogeneous, etc.) and strength of the binding interaction. But, these traces generally only are useful, if one knows in advance, the identity of the binding molecule 151 before it is introduced into the biochip. Label-free techniques generally cannot a priori identify molecules (unless of course the affinity of the initially tethered molecule is highly specific). Coupling NPOI with the affinity capture mass-spectroscopy approach described here allows for that identification.

The key to the approach is the additional capture capacity available because the biochip is porous. In what follows a calculation of that improvement will assume that the initial molecule tethered to the surface is a protein. However the analysis equally applies to other classes of molecules as well. As the wavelength of light used to probe the pores is generally the visible and near infrared (380-1,100 nm), the features caused by pores themselves and the biomolecules inside the pores are much smaller than the wavelength of light. This allows for simple linear addition of the various contributions to the refractive index inside the pores. Also, as the pores are reasonably homogenous throughout their length we can reasonably approximate the OPD signal to be:

OPD=2nL (1)

where n is the refractive index of the porous silicon layer, L is the length of that layer and the factor of 2 comes about because light passes through the layer twice. Now the porous silicon layer has two main components to it, the pore walls, which are silicon with refractive index n_{Si} and the pores themselves. The pores themselves also have two separate components, the buffer, with refractive index n_{buf} which fills the pores, and the protein with refractive index nₚᵣ, which is tethered to the sides of the pores. These three components may be added linearly as:

n=(1−P)n_{Si}+P[(1−fₚᵣ)n_{buf}+fₚᵣnₚᵣ] (2)

where P is the porosity (ie P=80% implies 80% of the volume is pores and 20% is pore walls) and fₚᵣ is the fraction of the pores occupied by protein (ie fₚᵣ=5% implies that 5% of the volume of the pores is occupied by protein, most of it tethered to the pore walls.

Again using the fact that all of the features inside the pores are smaller than the wavelength of light allows considering biomolecules, though they are separately bound to the pore walls, as forming a monolayer of an effective thickness. That is, if there were a single biomolecule, with an effective diameter of 5 nm (and therefore a volume of 65 nm³), bound in an area of 1,000 nm², for refractive index calculation purposes, this may be thought of as a monolayer over that area whose thickness is 0.065 nm thick. Considering now how this ‘effective monolayer’ thickness affects fₚᵣ this effective monolayer may be thought of as being everywhere along the pore wall. So if the pore wall has a radius of r, the area of the top of the pore is just that of a circle, πr². The area occupied by the effective monolayer is then:

π(r−dₚᵣ)² (3)

where dₚᵣ is the ‘effective thickness’ of that protein layer. Using Eqn. 3 and taking the average dₚᵣ throughout the length of the pore, fₚᵣ becomes:

$${f}_{pr} = \frac{π {r}^{2} - {π ( r - {d}_{pr} )}^{2}}{π {r}^{2}} \qquad ( 4 )$$

Now, considering the case where there is no protein in the pores, which would be that shown in FIG. 1 at the beginning of the immobilization 120, combining Eqn. 1 and Eqn. 2 and setting fₚᵣ to 0 gives:

OPDₒ=2[(1−P)n_{Si}+Pn_{buf}]L (5)

Porous silicon chips of the sort taught by Ervin et al. typically have L of 1,600 nm and P=80%. Using water as the buffer (n=1.33) this then gives an OPDₒ of approximately 5,800 nm as is typically seen and depicted in FIG. 1.

When biomolecule binds to the pores, this changes OPDₒ linearly in the amount of bound molecule. The change due to can be found by combining Eqn. 1 and Eqn. 2 and subtracting the initial signal in Eqn. 5:

ΔOPD=2P[fₚᵣnₚᵣ−fₚᵣn_{buf}]L (6)

ΔOPD therefore can be simply understood as upon binding a fractional area, within the pores increases its refractive index from that of buffer, to that of protein:

ΔOPD=2PfₚᵣL(nₚᵣ−n_{buf}) (7)

For a typical scenario (P=80%, L=1,600 nm, nₚᵣ=1.49, n_{buf}=1.33) so a 1 nm change in OPD corresponds to a fₚᵣ of 0.0024. Using Eqn. 4, which may be solved iteratively for dₚᵣ, then shows that in a typical scenario a ΔOPD=1 nm implies an ‘effective monolayer’ thickness dₚᵣ=0.049 nm.

So a 1 nm change in OPD signal corresponds to a 0.049 nm layer of biomolecule coating the entire insides of the pores. Of course, the coating is not even, but given that features are small than the wavelength of light it may be considered an even coating. Taking 1.1 g/mL as a typical density for a biomolecule, this implies that ΔOPD=1 nm corresponds to a surface coverage of ˜50 pg/mm² on the inside surface area of the pores.

In the context of AC-MS this implies a much greater capture capacity than is typical for surface plasmon resonance (SPR) which uses a planar sensor. Consider for a moment a planar sensor surface which is a square whose side, l, has length 80 nm. The surface area here is simply l² which equals 6,400 nm². Now if instead there were a pore in this 80 nm square with length 1,600 nm the total surface area still contains the initial 6,400 nm², partitioned between the pore wall and the bottom of the pore, but now the inside of the pore walls is added to the total area which in this case is simply the circumference of the circle formed by the pore (πl) times the length of the pore (L) which in the typical case (L=1,600 nm) implies an surface area of greater than 400,000 nm² or over 60 times the surface area available to a planar interferometer. This 60× factor will heretofore be referred to as the ‘surface area enrichment,’ which is given by the pores.

It is this surface area enrichment factor which allows the AC-MS application to be performed here. The details of the chip sizes will be described in the particular embodiments, but a typical AC-MS case would be the flow cell in a small molecule context. The flow cell has typically 2.0 mm² of sensor area, which given the typical surface enrichment factor, implies a total surface area of 120 mm². In the case of a small molecule experiment, one expects signal changes (ΔOPD) on the order of 0.5 nm (900). This then corresponds to 3 ng of small molecule captured by the flow cell chip.

As shown in 900, ˜2 ng of this 3 ng of material is eluted in the first 120 seconds. As typical flow rates are 20 μL/min this gives a final concentration of over 50 ng/mL in 40 μL of buffer. This may be readily characterized by direct infusion into a mass spectrometer, or a small amount of this material could be trapped at the beginning of the dissociation with a trap column then analyzed by LC-MS and then a small amount of material could be trapped at the end of the elution and analyzed by LC-MS to give a two point determination for k_{off}.

What follow are seven preferred embodiments of the invention that make use of different ionization source, different NPOI formats and different coupling schemes. The first four use an electrospray ionization source and a flow cell based NPOI instrument. The next embodiment uses a MALDI ionization source with a flow cell. The last two use a plate reader NPOI instrument. These embodiments generally involve an electrospray ionization source, but could use other types of ionization as well.

### Porous Silicon Chips

The porous silicon chips used in the seven embodiments described here are broadly similar. Nanopores with, typically 80 nm diameters, are etched into a typically 100-800 μm thick, silicon substrate typically about 1.5-2.0 μm deep. Light is reflected from the two refractive index interfaces, namely between the buffer, porous silicon interface and between the porous silicon, solid silicon interface. The optical path difference (OPD=twice the average refractive index times the physical length) between these two surfaces is measured in real time. A binding molecule is immobilized within the pores, affecting an OPD difference of e.g. 5,840−5,800=40 nm, 121 during the immobilization step. To this immobilized ligand, analyte is added causing an OPD difference of e.g. 5,860−5840=20 nm, 122 during the association step. Conditions are switched to dissociative and the analyte leaves the pores causing an OPD difference of 5,840−5,860=20 nm. The trace during this run could be indicative of any number of interactions between biomolecules, though generally as drawn shows an immobilized antibody binding and then unbinding to its antigen.

### First Preferred Embodiment

### Flow Cell with Electrospray and Fraction Collection

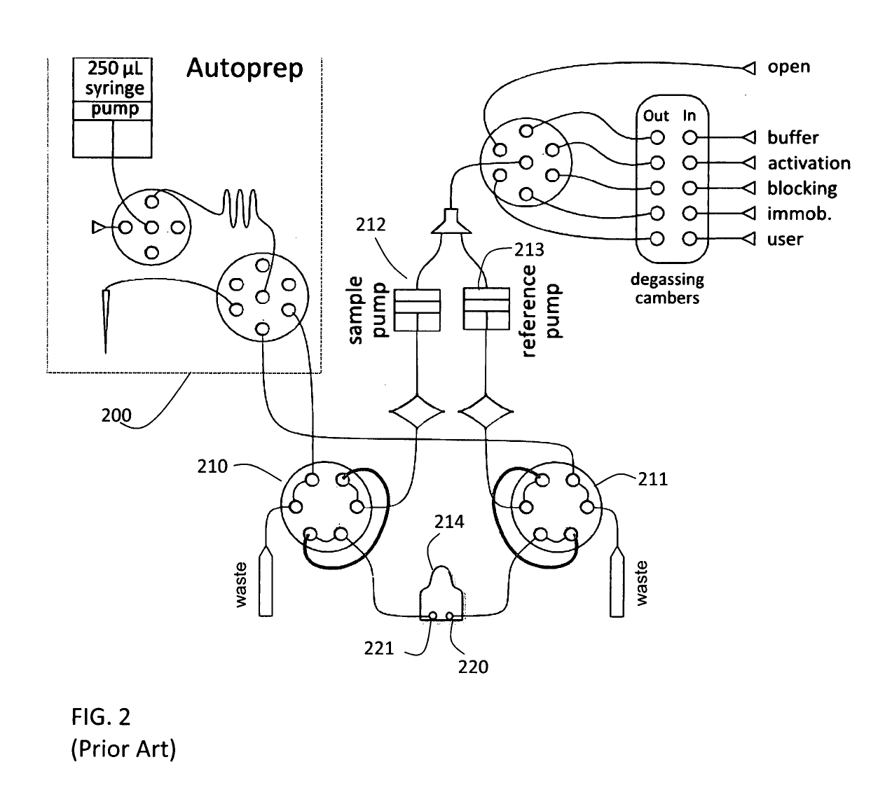

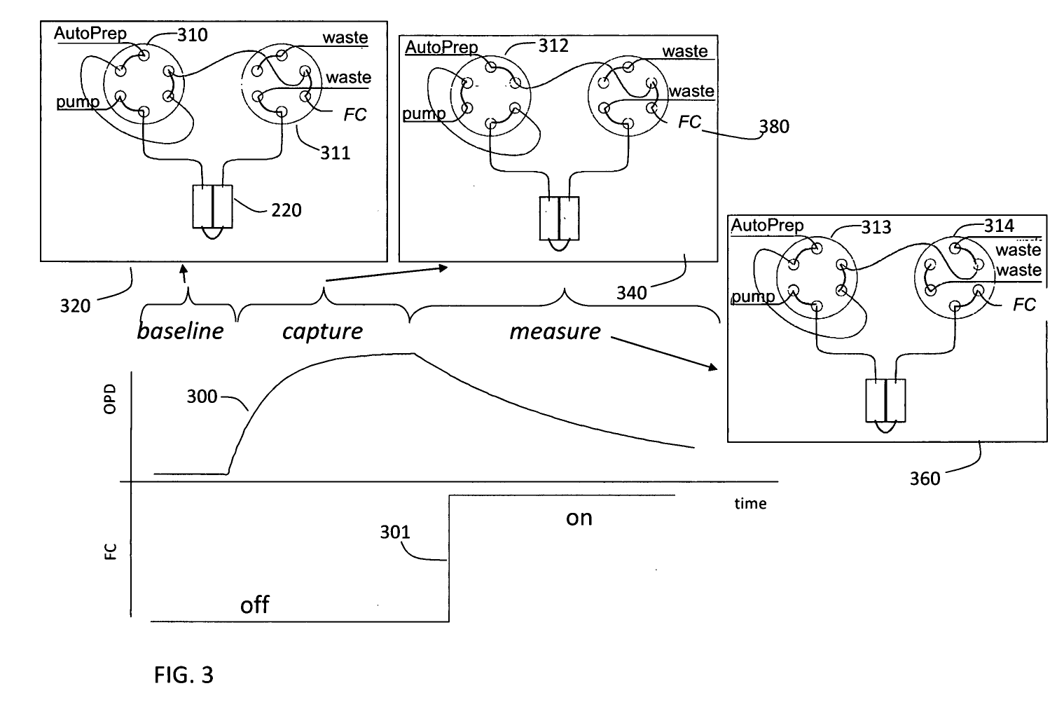

The first embodiment is sketched in FIG. 3. The NPOI flow cell instrument is used together with a fraction collector. This fraction collector could simply be a collection vessel of some sort. Initially capture molecule is placed on the porous silicon chip using a typical flow cell procedure. A carboxyl chip shown at 214 in FIG. 2, used for amino coupling, is used. This contains both a sample and reference channel 220 and 221. Using the AutoPrep 200 and two 6-port, 2-position injection valves, one for the sample 210 and one for the reference 211, solutions are introduced to the flow cell. Initially the chip is activated using water as running buffer, pumped by two separate pumps one for sample 212 and one for reference 213, at 8 μL/min. A solution of 200 mM, 1-ethyl-3-(3-dimethylaminopropyl) carbodiimide (EDC) and 50 mM sufo N-hydroxysuccinimide (sNHS) is used to activate the chip for 8 minutes. Then a solution of 20 μg/mL capture molecule is introduced in pH 5.0, 40 mM acetate buffer (1-1,000 μg/mL concentration may also be used). This immobilization continues for 12 minutes and causes OPD changes of between 5-50 nm during the immobilization step. Residual sulfo-succinimide esters on the surface are quenched using 1M, pH 8.5 ethanolamine for 5 minutes, and the running buffer is changed to pH 7.4 phosphate buffered saline (PBS) with 0.1% bovine serum albumin (BSA) over a 15 minute period. The chip is now ready for binding.

A schematic of the OPD signal during binding is shown 300 in FIG. 3. During the baseline phase 320 the AutoPrep is used to load the sample loop in the sample injection valve arranged as shown at 310. After the loop is loaded, the binding phase 340 may begin where the sample injection valve now routes the sample to the flow cell cartridge with the injection valve as shown at 312. During the association phase, the fraction collector (380) is not in line with the flow from the flow cell. After the binding completes, the sample injection valve switches back to having the sample loop off line as shown at 313 and the reference injection valve in the configuration as shown at 314 now routes the output to the fraction collector. As shown in the schematic the fraction collector turns off a bit after the dissociative phase begins as shown at 301. The fraction collected sample now contains whatever it was that bound to the binder initially placed on the cell.

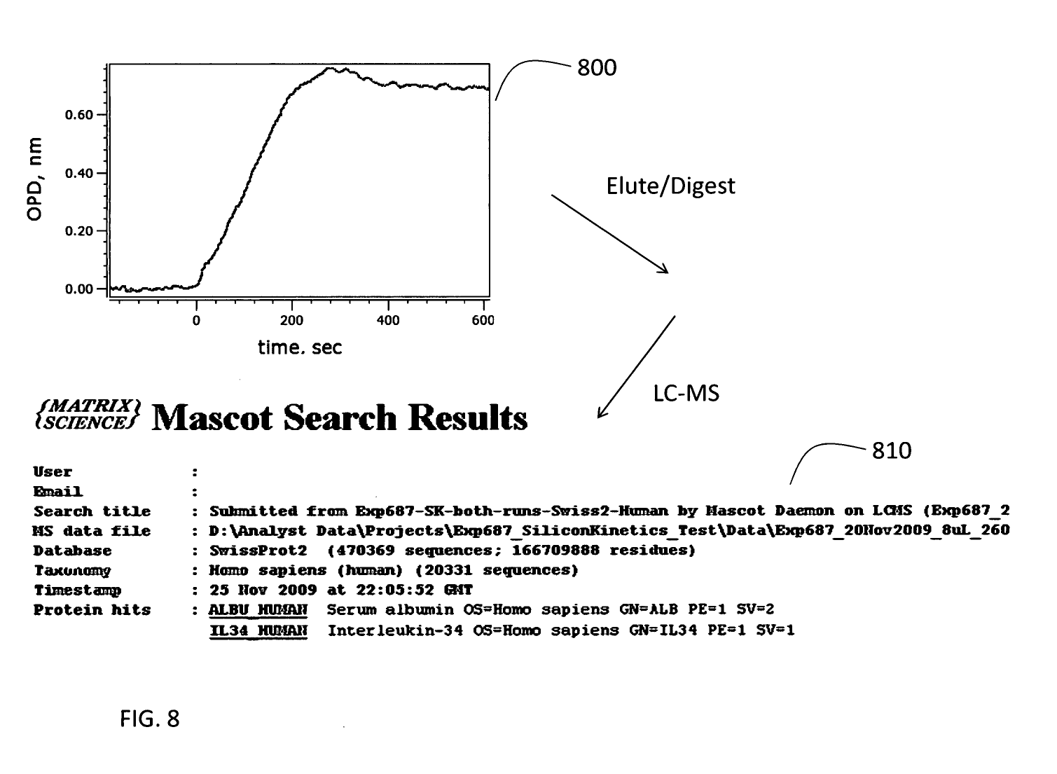

A specific example of such an approach is shown in FIG. 8 which is a print of a monitor display showing Mascot Search Results. Here, the an extra-cellular receptor for interleukin 34 (the ligand) is immobilized onto the flow cell chip as described at a concentration of 100 μg/mL. Interleukin 34 (IL34) (the analyte) is then doped into spent cell culture media at a concentration of 100 ng/mL. 50 μL of this material is then used during the binding portion of the experiment. The label-free binding signal shows a change in OPD of 0.7 nm between 0 and 240 seconds. This corresponds to 5 ng of IL34, corresponding to an almost complete capture of the material introduced. This material was then eluted and fraction collected using a 10 μL pulse of pH 2.2, 20 mM glycine. The collected fraction was neutralized with a 2× excess of 1M, pH 9.0 Tris. To this was added 10% acetonitrile and trypsin which was incubated overnight at 37° C. The result of this digestion were introduced into a capillary LC-MS system using an Agilent 1200 capillary LC (Agilent Corporation, Santa Clara, Calif.) with a 200 μM ID, 15 cm C18 column with 3 μm particles. Peptides were run using a standard gradient at 1 μL/min into a micro-flow electrospray ionization source (Microm Bioresources, Auburn, Calif.) into a 3200 QTrap from Applied Biosystems (formerly of Foster City, Calif.) operating in positive ion mode. The resulting data was analyzed using Mascot and a customized human protein database (810) show the successful identification of IL34.

### Second Preferred Embodiment

### Flow Cell with Electrospray and Direct Elution

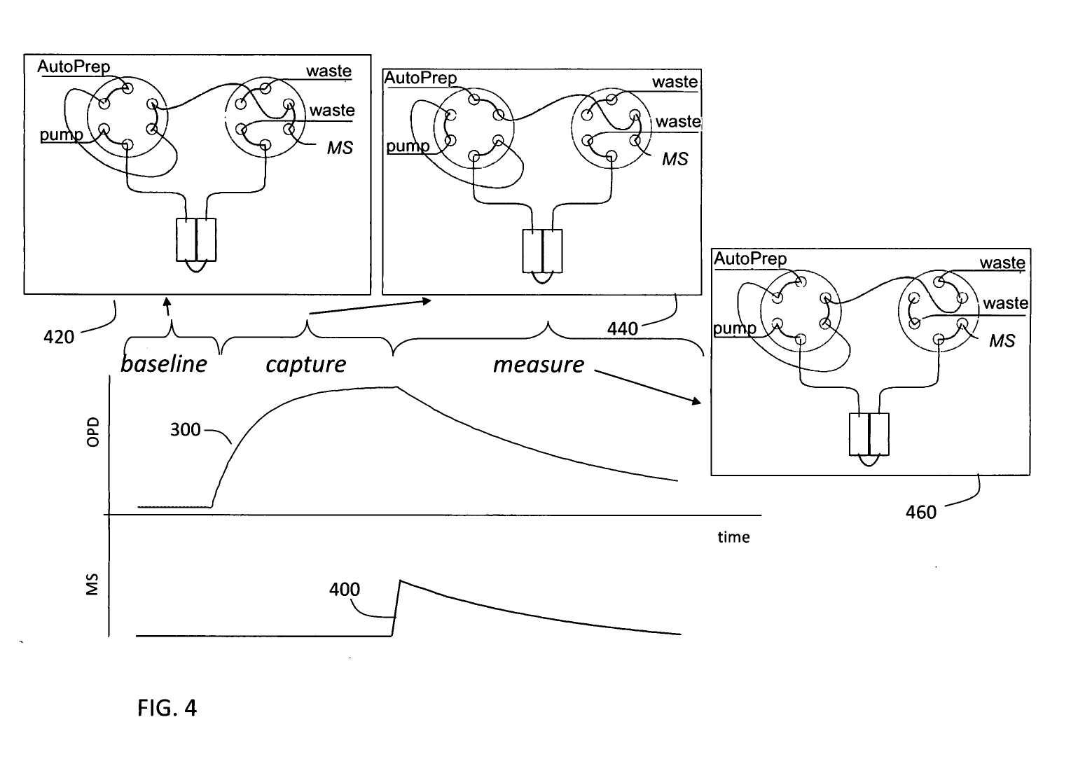

The second preferred embodiment is sketched in FIG. 4. Similar to the first embodiment, this second embodiment replaces the fraction collection output with an input directly into the mass spectrometer. Here, capture agent is immobilized as before and binding is initiated as before by a switch in the sample injection valve from the loading position with valves as shown at 420 to the inject position (440), however in this case binding buffers must be compatible with mass spectrometer ionization. The output to the mass spectrometer is sent into a y-union (not shown) in which a 0.1% formic acid in methanol mixture is added to the eluent from the flow cell device in order to aid electrospray ionization.

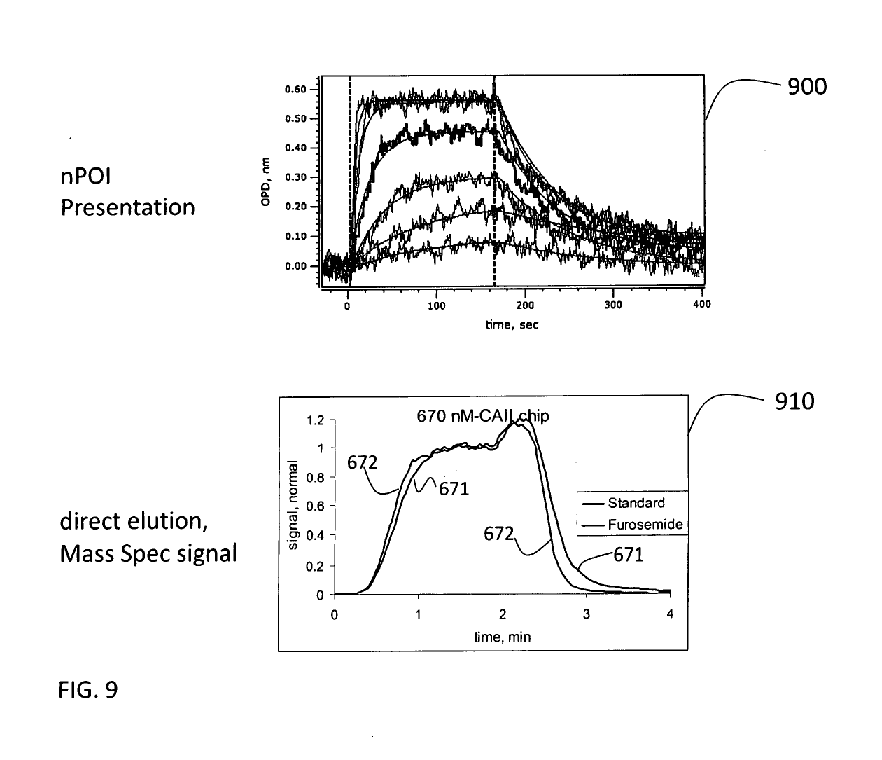

A specific example of such an approach is shown in FIG. 9. Here carbonic anhydrase II (CAII) (the ligand), an enzyme from bovine erythrocytes, is immobilized on the carboxyl chip at 100 μg/mL for 15 minutes. Furosemide (the analyte), is interacted with the carboxyl chip using the flow cell at five different concentrations between 10 nM and 10 μM. The label-free binding signal 900 from the NPOI is shown for several small molecule concentrations of the small molecule interacting with the immobilized protein.

To demonstrate the viability of AC-MS using the flow cell, direct elution and electrospray, hippuric acid and furosemide were analyzed for their electrospray ionization behavior using a Thermo LCQ Deca Ion-Trap Mass Spectrometer (Thermo-Fisher, Waltham, Mass.). They were found to ionize under similar conditions in 10%, 0.1% formic acid/methanol in 40 mM ammonium formate. Another CAII surface was prepared on a carboxyl chip by the identical means given 25 nm of immobilization density. A mixture of 670 nM furosemide and 670 nM of the hippuric acid standard were passed over the chip. directly eluting into a mass spectrometer during the entire binding run. The normalized total ion count of the furosemide 671 and hippuric acid (standard) 672 are plotted as a function of time 910. As can be seen the furosemide is delayed with respect to the hippuric acid due to its binding to the CAII showing that it is the strongest binder of the two.

Being able to clearly detect binding with direct elution is by far the most challenging of the electrospray embodiments and shows clearly how the affinity capture capability of the porous silicon chips allows for this invention. The two embodiments which follow make use of the concentration of analyte afforded with methods using LC-MS.

### Third Preferred Embodiment

### Flow Cell with Electrospray and LC Trap and Elute

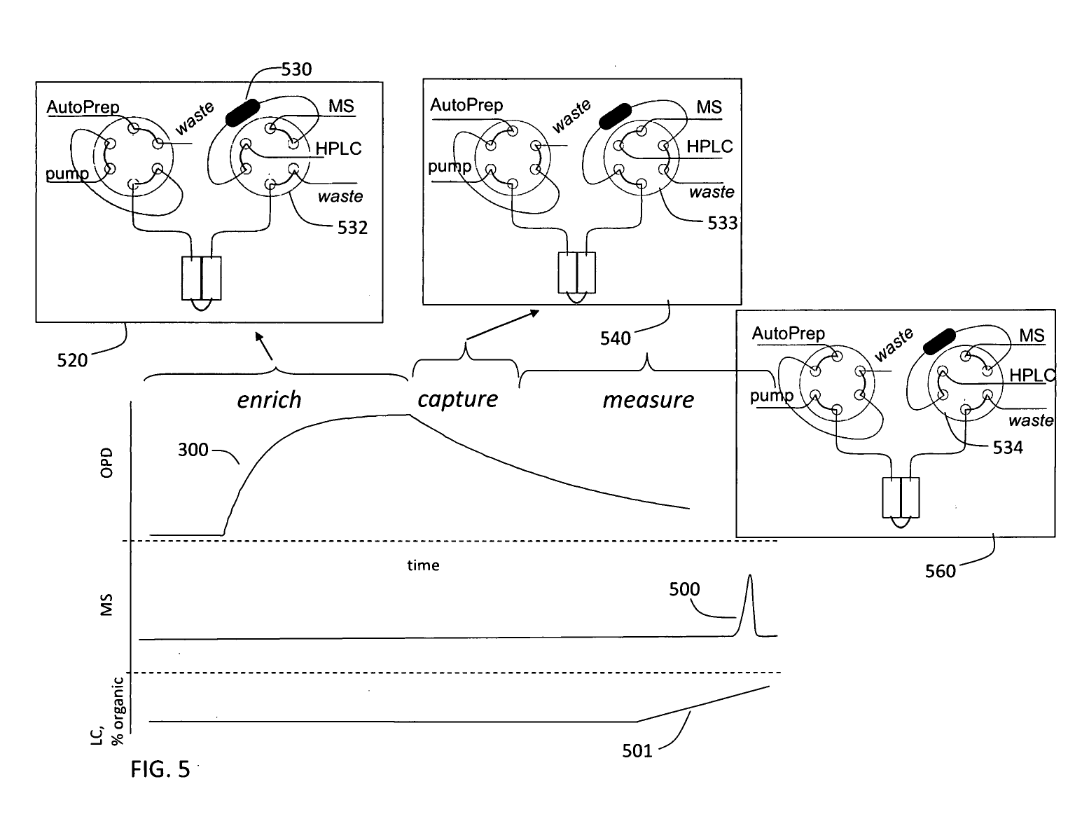

The third preferred embodiment is sketched in FIG. 5. This differs from the first and second embodiment in that a trap chromatography column 530 is placed on the reference side injection valves, a C18 packing is used in a 2.7 μM4 Opti-Pak trap from Optimize technologies (Oregon City, Oreg.). In this embodiment, the enrichment phase 520 proceeds as before with the sample injection valve sampling routing the flow cell eluent to waste while a liquid chromatography device (e.g. an Agilent 1100, Agilent Corporation, Santa Clara, Calif.) attached to the reference injection valve configured as shown at 532 flows water across the trap into a mass spectrometer (not shown). However, during capture, when conditions become dissociative, the reference injection valve configured as shown at 533 now switches to route the flow cell eluent onto the trap column. The trap column will then trap the reagent flowing across from the cell. This occurs for only a short time and then the reference valve switches back to placing the trap column in line with the LC-MS setup (534). The LC then begins its gradient to elute the material from the trap column onto the chromatography column (e.g. a Phenomenex Luna C18(2) phase, 3 μm particles, 2×10 mm column).

The LC device generally is flowing only a small percentage of organic (0-3%) during its run, but the gradient picks up dramatically during the measure phase (501) which causes a peak to appear when the affinity capture molecule or molecules come out (500). The valves on the NPOI instrument taught by Ervin et al. (patent application Ser. No. 12/221,119), unlike the vast majority, if not all SPR instruments, contains valves which allow for 5,000 psi pressure rating, so are completely compatible with HPLC whereas most SPR valving fails at 100 psi.

By allowing this combination of trapping the previously affinity captured molecules, an LC-MS separation can be performed which not only concentrates sample in the MS signal (501), but allows for separations of several small molecules. This allows many molecules to be initially characterized. For instance, a mixture of 10s to 100s of small molecules or peptides, could be simultaneously introduced into this affinity capture mass spectrometry embodiment. If several of them were to bind, as might be expected if several had reasonably similar on-rates, then these unknowns could all be more readily identified using a chromatography step as this chromatographic separation is known to decrease ion suppression.

### Fourth Preferred Embodiment

### Flow Cell with Electrospray and Time Resolved LC Trap and Elute

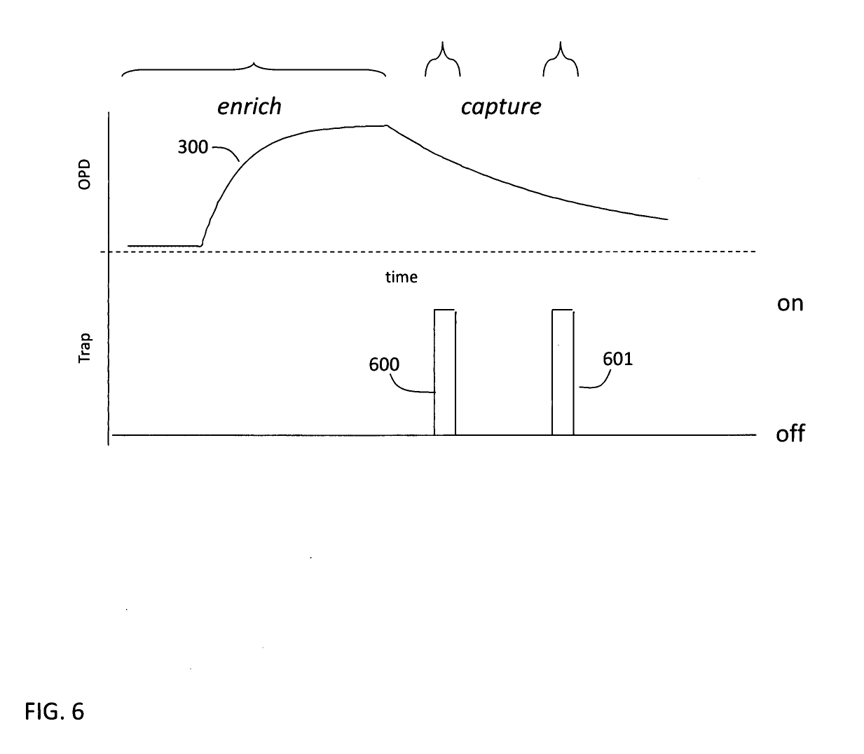

The fourth preferred embodiment is an extrapolation of either the first or third embodiment and is shown in FIG. 6. The enrichment and disassociation curve is shown at 600. Here fraction collection, or trapping with LC-MS elution, is performed at fixed times after the dissociation reaction is initiated. By characterizing the type and amount of material at a small set of known times an approximation to the off rate made be made.

Considering this embodiment in the context of the CAII/furosemide experiment discussed above (900), fractions could be collected from say 180-200 seconds and from 280-300 seconds as shown at 600 and 601. At the 20 μL/min flow rate used here these 20 seconds fraction collected or trapped fractions would correspond to about 7 μL of sample at a concentration ranging between about 5-50 ng/mL. Assuming the analytes are reasonably ionizable, these amounts and concentrations could be readily characterized by a mass spectrometer, even if the molecules are not known ahead of time.

If the LC-MS set were fast enough, these time resolved slices could be analyzed in a single run. If not, repetitive runs at the same concentration could be used and the AC-MS detected, dissociation curve could be reconstructed from the several measurements which were separated in time. Normalization to the detected NPOI signal, which is measured each time in parallel, could improve the resolution of this.

### Fifth Preferred Embodiment

### Flow Cell with MALDI and Time Resolved Spotting

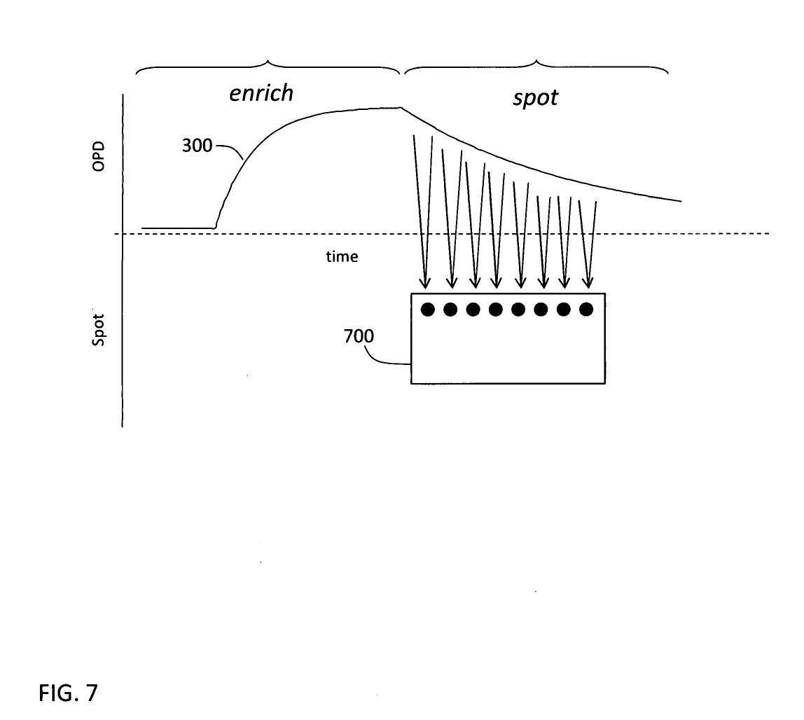

The previous four embodiments addressed the utilizing an AC-MS technique with an electrospray source on a mass spectrometer. In this fifth preferred embodiment, shown in FIG. 7, AC-MS is used together with a MALDI spotter. Here the schematic of the binding reaction is again shown (300) but now, during the dissociation phase, the output is sent to a MALDI spotting system (e.g. the Probot MALDI spotter from Dionex Corporation, Sunnyvale, Calif.) which then spots the output on a MALDI plate using 2,5-dihydroxy benzoic acid (DHB) as the absorbing matrix. The flow rate in this embodiment so that spots on the plate correspond to 1 μl of volume or 3 seconds of time. The plate could then be analyzed with a commercially available 4700 Proteomics Analyzer MALDI tof/tof (Applied Biosystems, formerly of Foster City, Calif.).

Spots are the MALDI plate correspond to time of dissociation. Here peptide libraries could be introduced to an unknown binder and then used this embodiment to efficiently characterize the off rates of bound peptides by reconstructing the time resolved data from the MALDI spots.

### Well Strips

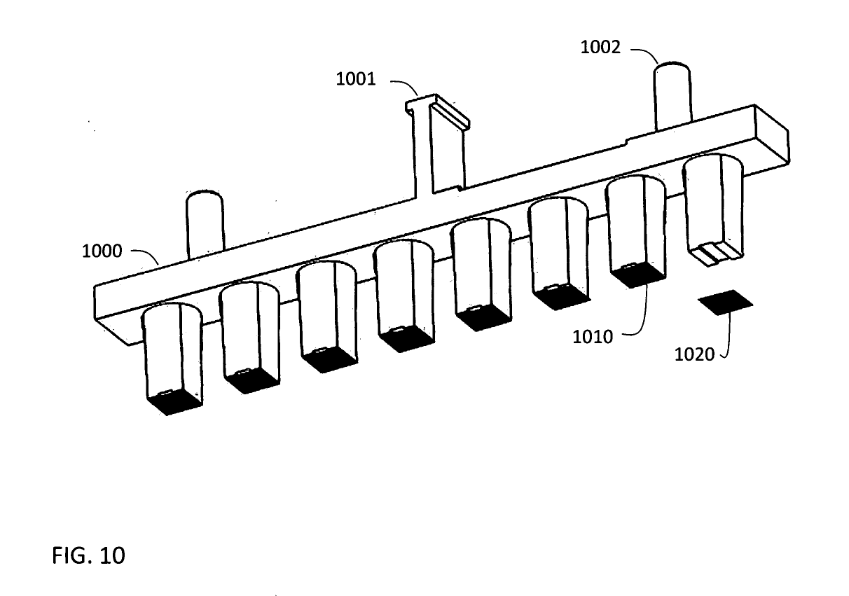

The previous five embodiments addressed the coupling of AC-MS with a flow cell based NPOI instrument. The final two embodiments use the plate reader mode of an NPOI instrument as taught by Ervin et al. (U.S. patent application Ser. No. 12/221,119). FIG. 10 shows the mounting of the porous silicon biochips in these last two embodiments. This ‘well strip’ 1000 is piece formed typically by injection molding and has eight posts on it which hold porous silicon biochips ranging in size from 1.0-5.2 mm squares but here typically 2.6 mm squares. These posts are placed to match the pitch in the wells of a 96 well plates, 9 mm by SBS format. With smaller posts well strips can also be used in 384 well plates, which have a 4.5 mm pitch.

Porous silicon biochips (1020) are placed onto the ends (1010) of these posts using an adhesive like glue, double sided tape, or adhesive transfer tape. The well strips are then placed into the well plates using their handle (1001) by either manual or automated means. Posts on the well strips (1002) are used by automated equipment to sense whether a well strip is present. Well strips, when used to acquire an NPOI signal, are placed into micro-titer well plates which have an optically transparent bottom so that the reflection off of the well strips may be monitored.

### Sixth Preferred Embodiment

### Plate Reader with Electrospray

In this sixth preferred embodiment a microtiter well plate is prepared with columns having different fluids. Well strips are then placed in a column for a certain amount of time in order to affect a chemical change or wash step on the porous silicon chips, while NPOI data are acquired from underneath the microtiter plate. The general idea is to use bait protein (the ligand) attached to the porous silicon chips at the end of the well strips to bind a prey molecule (the analyte) in one column and then bring that bound protein into a dissociative solution in another column. This solution is then tested by electrospray LC-MS to see what, if anything has bound.

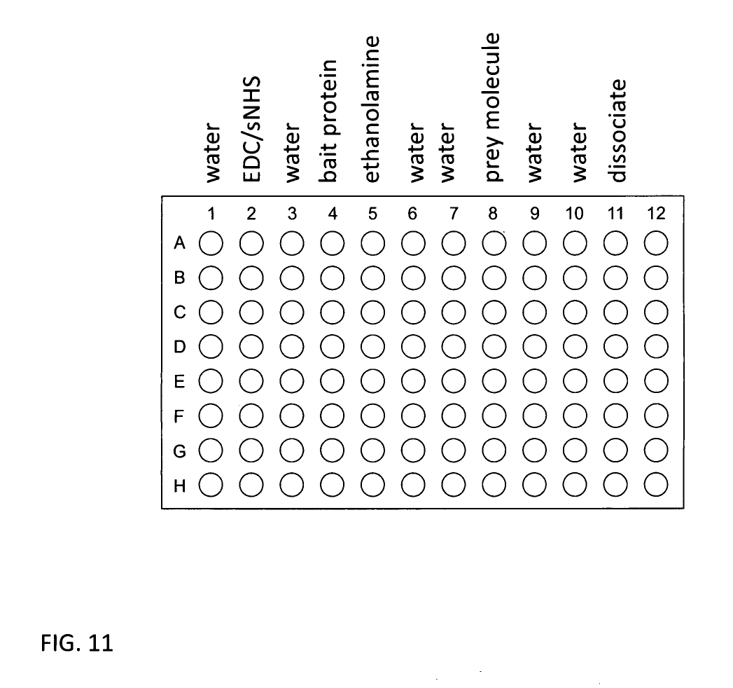

FIG. 11 gives an example of how a 96 well microtiter plate would be set up to allow AC-MS using an NPOI plate reader with carboxyl chips. 100 μL of solution would be placed in each well of the microtiter well plate as shown in FIG. 11. The well strip is first placed in column 1 and equilibrated in water for 20 minutes. The well strip is then moved to column 2, which contains 200 mM EDC/50 mM sNHS which reacts with the well strips for 20 minutes. The well strip is then placed in water in column 3 for 5 minutes to wash. These activated strips are reacted then with the bait protein in column 4. Typically, this is at pH 4.5-6.0 at a concentration of 1-1,000 μg/mL. The immobilization here is allowed to proceed for 30 minutes. The NPOI signal is checked to ensure that an appropriate level of bait protein immobilization has been reached (typically 2-60 nm). The activated chip surfaces are then quenched by moving to column 5 using 1M, pH 8.5 ethanolamine for 12 minutes. The well strips are then moved to be washed, first for five minutes in column 6 and then for five minutes in column 7. Prepared strips are then interacted with potential prey molecule, typically 0.1-1,000 μg/mL in PBS in column 8 for 20 minutes. The strips are then washed for 5 minutes in column 9 and then washed again for 5 minutes in column 10. Column 11 is setup to be a dissociative buffer, for instance using pH 2.2, 40 mM glycine. The strips are placed here for 10 minutes and then removed.

For those samples which showed an appreciable NPOI binding during the binding while in column 8 with the prey molecule, the corresponding dissociation well is then analyzed by electrospray LC-MS. This can then determine whether something bound and then determine what that might be. If the prey molecules were proteins then these could first be digested off line before the LC-MS step.

The 2.6 mm chips used with this embodiment have a total area of 6.8 mm². Using the furosomeide/CAII experiment (900) as a guide for the expected signal in this embodiment, the expected 0.5 nm of OPD change corresponds to 25 pg/mm² which corresponds to ˜10 ng. Assuming that half of this is washed away in the wash steps in columns 9 and 10, this still leaves 5 ng of material in the 100 μL of column 11. This 50 ng/mL can be readily detected by electrospray mass spectroscopy assuming the prey molecule can be readily ionized. Of course, there needs to be enough prey material available in the wells of column 8 to saturate the chip.

One skilled in the art will realize they are numerous other arrangements of the microtiter well-plate other than that shown in FIG. 11 which will allow for AC-MS in the context of this embodiment, including for instance reuse of a prepared well strip with or without regeneration.

### Seventh Preferred Embodiment

### Plate Reader with Electrospray without NPOI Data

In this seventh preferred embodiment a plate reader type setup is coupled with electrospray mass spectroscopy, but here NPOI data are not taken, otherwise the procedure is similar to that of the sixth embodiment. The well strips are used without the analysis provided by the OPD signal, in identically the same way as described before, except all wells in column 11 are analyzed by LC-MS. The advantage conveyed by knowing whether there was binding as shown by the NPOI signal, and that whether the LC-MS experiments can in fact identify binders, is not present in this embodiment.

### VARIATIONS

While there have been shown what are presently considered to be preferred embodiments of the present invention, it will be apparent to those skilled in the art that various other changes and modifications can be made herein without departing from the scope and spirit of the invention. Therefore, the scope of the invention should be determined by the appended claims and their legal equivalents and not by the examples that have been given.

What is claimed is:

1. A process for making label-free molecular ligand-analyte binding measurements comprising the steps of:
A) providing a label-free porous silicon based optical sensor comprising:
a) a spectrometer based optical system comprising at least one light source and two spectrometers,
b) at least one porous silicon region comprising at least 1000 pores, each pore having a nominal width and a nominal depth at least 10 times larger than said nominal width with the depth of at least most of the pores in said porous silicon region defining a top surface and a bottom surface being parallel or approximately parallel to the top surface, said porous silicon region being adapted to permit molecular binding interactions,
c) a computer processor adapted to produce optical path difference information based on output from each of said two spectrometers,
B) providing a mass spectrometer,
C) depositing in a plurality of said at least 1000 pores a fluid containing a ligand chosen to attach to walls of said plurality of said at least 1000 pores,
D) depositing in a said plurality of said at least 1000 pores a fluid containing a buffer and an analyte containing fluid,
E) utilizing the label-free porous silicon based optical sensor to monitor the molecular binding interactions of said ligands and analytes in said analyte containing fluid deposited in said plurality of said at least 1000 pores,
F) separating at least a portion of said buffer from at least a portion of said analytes and analyze with said mass spectrometer said analytes so separated at least one time increment,
G) using information from said optical sensor and said mass spectrometer to characterize molecular binding and disassociation reactions between said analytes and said ligands.

2. The process as in claim 1 wherein said analyte containing fluid contains a plurality of different analyte and information obtained from said mass spectroscope is utilized to identify specific analytes involved in the binding and disassociation reactions.

3. The process as in claim 1 wherein said label-free porous silicon based optical sensor comprises:
A) a single porous silicon flow cell unit,
B) a multiple porous silicon flow cell unit,
C) a micro-well plate adapted to hold a porous silicon chip in a plurality of micro wells,
D) one or more exchangeable format trays adapted to position said single porous silicon flow cell unit, said multiple porous silicon cell unit and said micro-well plate serially within said base unit,
E) a plurality of fluid systems adapted to provide fluids containing buffer solutions, ligand containing solutions, and analyte containing solutions to said single flow cell, said multiple flow cell unit and said micro-well plate, and F) a control system comprising a computer processor adapted to provide automatic optical analysis serially of molecular interactions within the porous silicon chips in said single flow cell, said multiple flow cell or said micro-well plate, depending on which of the three formats is being.

4. The process as in claim 1 wherein said label-free porous silicon based optical sensor is adapted for utilization of porous silicon chips positioned in micro-well plates.

5. The process as in claim 1 wherein said label-free porous silicon based optical sensor is adapted for utilization of porous silicon chips positioned in flow cells.

6. The process as in claim 1 wherein said label-free porous silicon based optical sensor is adapted for utilization of porous silicon chips positioned in flow cells and includes a fraction collector to which output of the flow cells are directed during at least a portion of a period of disassociation phase.

7. The process as in claim 6 wherein the flow to the fraction collector is limited to a desired period soon after the disassociation phase begins so as to collect a sample containing whatever it was that bound to the ligand.

8. The process as in claim 7 wherein the collected sample is analyzed in an electrospray mass spectroscope.

9. The process as in claim 5 wherein a portion or all of output flow from the flow cells is directed to the mass spectroscope for analysis as a function of time.

10. The process as in claim 9 wherein the mass spectroscope in an electrospray spectrometer and a chemical chosen to aid in the electrospray ionization is added to the output flow directed to the mass spectroscope.

11. The process as in claim 5 wherein the label-free porous silicon based optical sensor is equipped with a trap chromatography column adapted to trap portions of fluid flowing from the flow cells.

12. The process as in claim 11 wherein molecules trapped in the trap chromatography are subsequently analyzed with the mass spectroscope permitting simultaneous analysis of many small molecules or peptides.

13. The process as in claim 6 wherein fraction collection is performed at fixed times after disassociation begins.

14. The process as in claim 5 wherein the output of the flow cells are sent to a MALDI spotter for analysis.

15. The process as in claim 4 wherein a well strip comprising posts adapted for the mounting thereon of porous silicon biochips is used to sequentially submerge the biochips into micro-wells in the micro-well plate containing buffer fluid, fluid containing ligands and fluid containing analytes to produce the binding and disassociation reactions.

16. The process as in claim 15 wherein an optical path difference measurement is not performed.
